(19) United States
(12) Patent Application Publication
(10) Pub. No.: US 2008/0281422 A1
(43) Pub. Date: Nov. 13, 2008

(54) ARTHROSCOPIC KNOTLESS TECHNIQUE FOR COLLAGEN PATCH FIXATION

(76) Inventor: Reinhold Schmieding, Naples, FL (US)

Correspondence Address: DICKSTEIN SHAPIRO LLP, 1825 EYE STREET NW, Washington, DC, 20006-5403, US

(21) Appl. No.: 12/113,879

(22) Filed: May 1, 2008

Related U.S. Application Data
(60) Provisional application No. 60/915,290, filed on May 1, 2007.

(51) Int. Cl. A61F 2/28; A61B 17/04; D04G 5/00

(52) U.S. Cl. 623/16.11; 606/228; 289/1.2

(57) ABSTRACT

An arthroscopic method of resurfacing with a collagen patch. A collagen patch is prepared by dimensioning the patch to correspond to the articular surface, and by providing a series of loops installed along the edge of the patch. The loops correspond to holes on the articular surface. Once the patch has been successfully placed on the surface, the loops are positioned in close proximity to the pre-drilled holes. Fixation devices such as anchors may be installed over the loops, to attach the perimeter of the graft to the surface through the pre-drilled holes.

This application claims the benefit of U.S. Provisional Application Serial No. 60/915,290 filed on May 1, 2007, the entire disclosure of which is incorporated by reference herein.

### FIELD OF THE INVENTION

The present invention relates to methods and instruments for arthroscopic surgery and, more specifically, to methods and instruments for securing a replacement articular surface patch.

### BACKGROUND OF THE INVENTION

When soft tissue such as a ligament or a tendon becomes detached from a bone, surgery is usually required to reattach or reconstruct the tissue. Often, a tissue graft is attached to the bone to facilitate regrowth and permanent attachment. Various fixation devices, including sutures, screws, staples, wedges, and plugs have been used in the past to secure soft tissue to bone. For example, in typical interference screw fixation, the tissue (for example, soft tissue) is fixed to the bone by driving the screw into a blind hole or a tunnel in the bone while trapping the end of the tissue between the screw and the bone tunnel.

Various approaches to joint repair and graft surface replacement are known in the art. For example, U.S. Patent Publication No. 2006/0149370, the disclosure of which is incorporated by reference herein, describes a method and apparatus for arthroscopic joint resurfacing. Additional, simple arthroscopic joint surface repair techniques are desirable.

### SUMMARY OF THE INVENTION

The present invention provides apparatus and methods of arthroscopic knotless technique for patch fixation (for example, collagen patch fixation). In an exemplary embodiment, the invention provides a patch preferably comprising collagen that is sized appropriately to cover the articular surface being repaired. The patch is sized to dimensions that allow it to be disposed into a tubular configuration for easy introduction to the joint space. A series of loops (for example, suture loops) are installed along or around the edge or periphery of the patch. The patch is inserted through a cannula having distal slots designed to hold the loops in place. Once the patch is introduced into the joint space, the suture loops are secured to bone using fixation devices such as bone anchors.

The present invention also provides a method of knotless technique for patch fixation during surgery (for example, during arthroscopy). The method of the present invention comprises the steps of: (i) providing at least one loop (for example, suture loop) around or along a periphery of a patch (for example, collagen patch); (ii) inserting the patch into a cannula (for example, arthroscopic cannula); (iii) subsequently, inserting the patch into the joint; and (iv) securing at least one loop of the patch with at least one knotless fixation device (for example, an Arthrex V-Tak™ anchor, as disclosed in U.S. Patent Publication No. 2005/0080455, or an Arthrex PushLock™ anchor, as disclosed in U.S. Patent Publication No. 2004/0093031) into a hole (for example, a predrilled hole) in the bone.

Other features and advantages of the present invention will become apparent from the following description of exemplary embodiments of the invention with reference to the accompanying drawings.

### BRIEF DESCRIPTION OF THE DRAWINGS

FIG. 1 is a lateral view of a human shoulder with the glenoid undergoing an exemplary method of glenoid resurfacing according to the present invention;

FIG. 2 illustrates the glenoid of FIG. 1 at a preparation stage subsequent to that shown in FIG. 1;

FIG. 3 illustrates a collagen patch prepared in accordance with the present invention;

FIG. 4 illustrates the collagen patch of FIG. 3 at a preparation stage subsequent to that shown in FIG. 3;

FIG. 5 illustrates the collagen patch of FIG. 4 in the proximity of the glenoid of FIG. 2 undergoing a resurfacing technique according to the present invention;

FIG. 6 illustrates the collagen patch of FIG. 4 in the proximity of the glenoid of FIG. 2 and at a stage subsequent to that shown in FIG. 5; and

FIG. 7 illustrates the collagen patch of FIG. 4 in the proximity of the glenoid of FIG. 2 and at a stage subsequent to that shown in FIG. 6.

### DETAILED DESCRIPTION OF THE PREFERRED EMBODIMENTS

The following description is provided to enable a person skilled in the art to make and use the invention and sets forth the best modes contemplated by the inventors of carrying out their invention. Various modifications, however, will remain readily apparent to those skilled in the art.

Although the present invention will be described below with reference to an exemplary glenoid repair, the invention is not limited to this exemplary embodiment. Accordingly, the structures and techniques of the present invention have applicability to joint repairs generally, and the invention must not be limited to glenoid repairs. Thus, the invention has applicability to any bearing surfaces in joints which are replaced or resurfaced arthroscopically using a patch secured in place with suture.

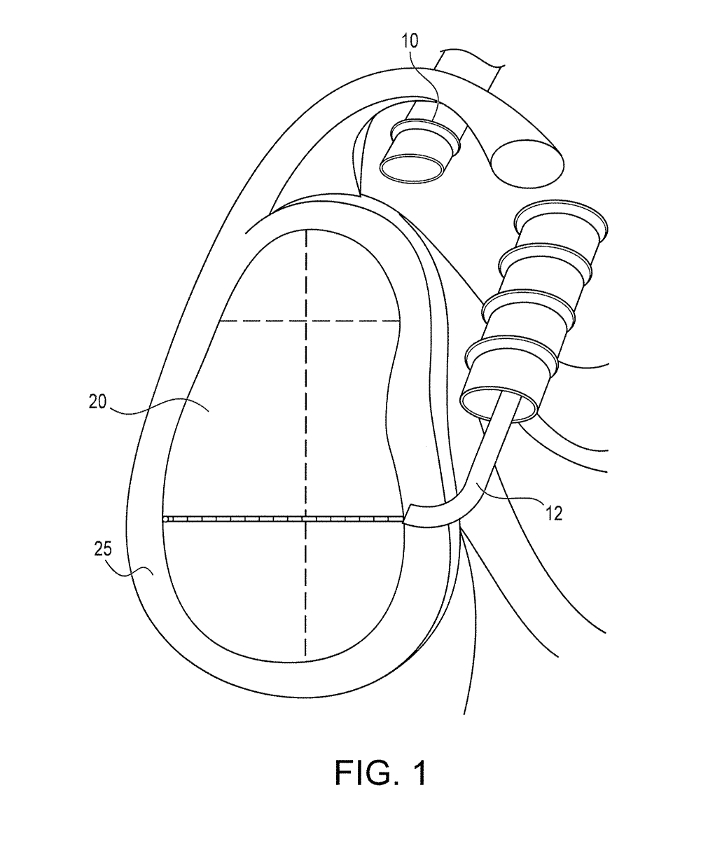

Referring now to the drawings, where like elements are designated by like reference numerals, FIGS. 1-7 illustrate a method and apparatus of arthroscopic knotless technique for collagen patch fixation.

Bearing surfaces in joints, such as a human shoulder joint, may be replaced or resurfaced arthroscopically using a patch secured in place with suture. The securing suture preferably includes multiple loops of a high strength suture. Exemplary high strength suture materials include, without limitation, those that are based on polyethylene. These suture materials typically contain filaments, fibers, or threads made in whole or in part from a polyethylene. A preferred example of a high strength suture is FiberWire™ suture, sold by Arthrex, Inc. of Naples, Fla., and described in U.S. Pat. No. 6,716,234, the disclosure of which is incorporated herein by reference.

The high strength suture is formed into loops. Loops can be formed by fabricating suture into continuous loops. The suture loops are secured preferably around the periphery of the patch, for example, by piercing the suture through the patch. The suture loops are fastened to bone using bone anchors to hold the patch in place proximate the joint surface being repaired.

The patch is preferably a collagen patch sized appropriately to cover the articular surface being repaired. The collagen patch is capable of being disposed into a tubular configuration for easy introduction to the joint space. In an exemplary embodiment, a bovine collagen patch, optionally treated to promote joint development and bone growth, is inserted through a cannula having distal slots to hold suture loops in place. The patch is subsequently introduced into the joint space. The suture is formed into loops, which are secured to bone using fixation devices such as bone anchors. Attachment of the suture to the patch and forming the suture into loops may take place prior to insertion of the patch into the joint space. Further, the suture loops may be formed prior to their attachment to the patch.

As described below, the loops will be captured by using devices such as individual forked anchors, for example V-Tak™ anchors, sold by Arthrex, Inc. of Naples, Fla. and described in U.S. Patent Publ. No 2005/0080455 A1 (the disclosure of which is herein incorporated by reference), allowing attachment of the entire perimeter of the patch to the glenoid.

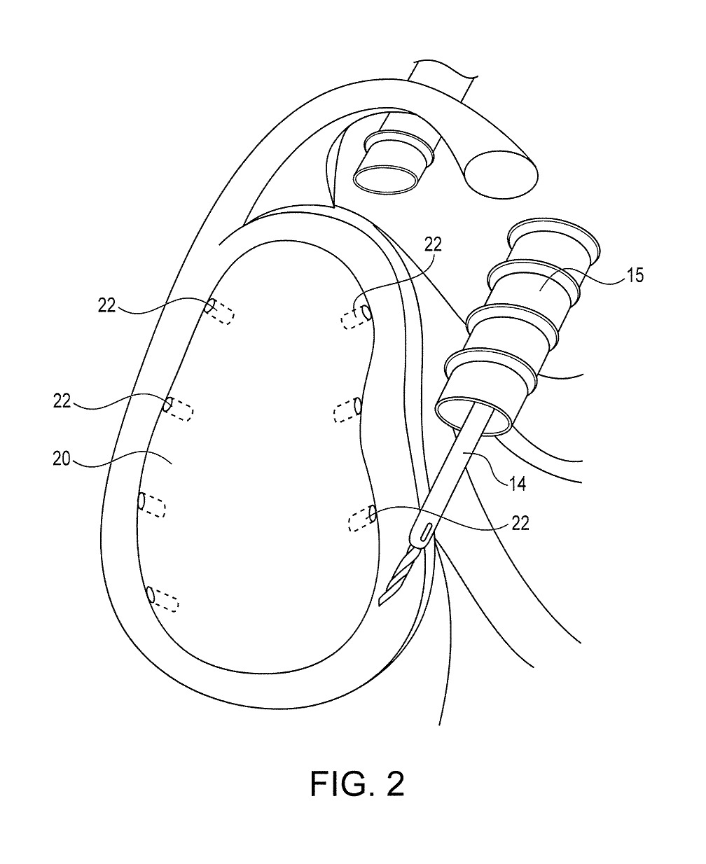

An exemplary method of secured fixation of tissue to bone using a suture anchor is illustrated in FIGS. 1-7. FIGS. 1 and 2 illustrate the interior of a right human shoulder in a lateral perspective with glenoid 20 and glenoidal labrum 25 that will accommodate a collagen patch 100 in accordance with an exemplary method of glenoid resurfacing of the present invention.

Prior to the method of graft fixation of the present invention, appropriate radiological studies may be conducted to determine if the humeral head and/or glenoid 20 have advanced patterns of wear that may require procedures alternative or additional to the one of the present invention. For example, excessive glenoid wear or severe humeral head deformity with advanced osteophytic formation may require more aggressive procedures.

The method of glenoid resurfacing of the present invention may be performed in the lateral decubitus or beach chair position. The arthroscope is initially inserted into the glenohumeral joint through a posterior portal. Preferably, this portal is placed 3 cm distal to the posterior corner of the acromion, in a position lateral and inferior to the standard posterior portal of the shoulder. The lateral placement facilitates proper anchor placement into the glenoid rim. The inferior placement allows access to the inferior capsule and labrum as well as the posterior/inferior glenoid rim.

An anterior inferior portal is created with an outside-in technique just superior to the articular fibers of the subscapularis tendon and lateral enough to provide the proper angle for anchor placement into the glenoid rim. A 8.25 mm threaded cannula is placed into this portal. Initial debridement of the joint may be performed with a mechanical shaver through this portal, to enable complete visualization of the glenoid surface.

Once the complete visualization of the glenoid is established, the glenoid dimensions are measured using arthroscopic measuring probes. Preferably, three measurements may be conducted, including total glenoid height, superior ⅓ width and the widest point. As shown in FIG. 1, two measuring probes are utilized. The straight probe (which would be introduced through cannula 10 of FIG. 1) is utilized to obtain the anterior to posterior width of the glenoid at the superior ⅓ of the glenoid and at the widest point. The right angle measuring probe 12 is utilized through the anterior portal to obtain superior to inferior measurements (the total glenoid height).

Alternatively, an antero-superior portal may be created with an outside-in technique. This portal may be utilized for visualization during the procedure. The straight measuring probe may be utilized to obtain the glenoid height through this portal prior to switching the arthroscope to this position. After all measurements are obtained, the results may be recorded and the arthroscope placed through the antero-superior portal, so that another 8.25 mm threaded cannula is placed in the posterior portal.

Preparation of the glenoid surface is conducted by removing any remaining articular cartilage using a combination of a rasp, curette and mechanical burr, for example. The bony surface around the edge of the glenoid adjacent to the labrum is then gently debrided to reveal a bleeding bed. Care is taken to preserve any labral tissue that remains, as it will be utilized to assist in fixation of the allograft in the procedure.

Once the anterior and posterior rims of the glenoid are defined, holes 22 (FIG. 2) may be formed around the perimeter of the glenoid 20 using a drill 14 and a guide 15 (FIG. 2), for example, a V-Tak drill and a Bio-SutureTak drill guide, both sold by Arthrex, Inc. of Naples, Fla. Holes 22 are placed around the anterior and posterior perimeter of the glenoid rim just as they would be placed in a labral repair immediately adjacent to the glenoid face. The orientation of the longitudinal axis of the holes relative to the longitudinal axis of the glenoid 20 may vary, depending on the characteristics of the glenoid, such as the size and configuration, as well as the accessibility to the glenoid by the surgeon manipulating the drill 14. Thus, the holes 22 may have a parallel orientation relative to each other, or alternatively, may be orientated at various angles with respect to each other.

In an exemplary embodiment, and as shown in FIG. 2, drill holes 22 are placed four anterior and four posterior, and equally spaced from each other. Thus, although FIG. 2 shows the formation of eight holes drilled within the glenoid 20, the invention is not limited to this exemplary embodiment, and contemplates the formation of any number of holes within the glenoid rim with any configuration and orientation, depending upon the configuration of the glenoid and of the repair site, as well as the configuration of the drilling instrument and of the collagen patch to be subsequently attached.

Subsequent to the formation of the holes 22 and prior to the collagen patch insertion, capsular release could be performed circumferentially, to help increase motion that is usually compromised in the arthritic shoulder.

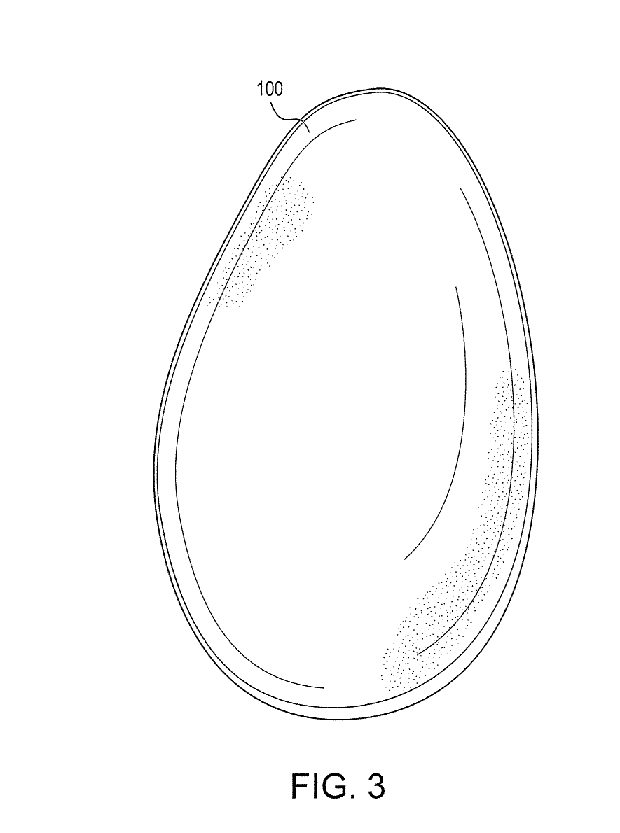

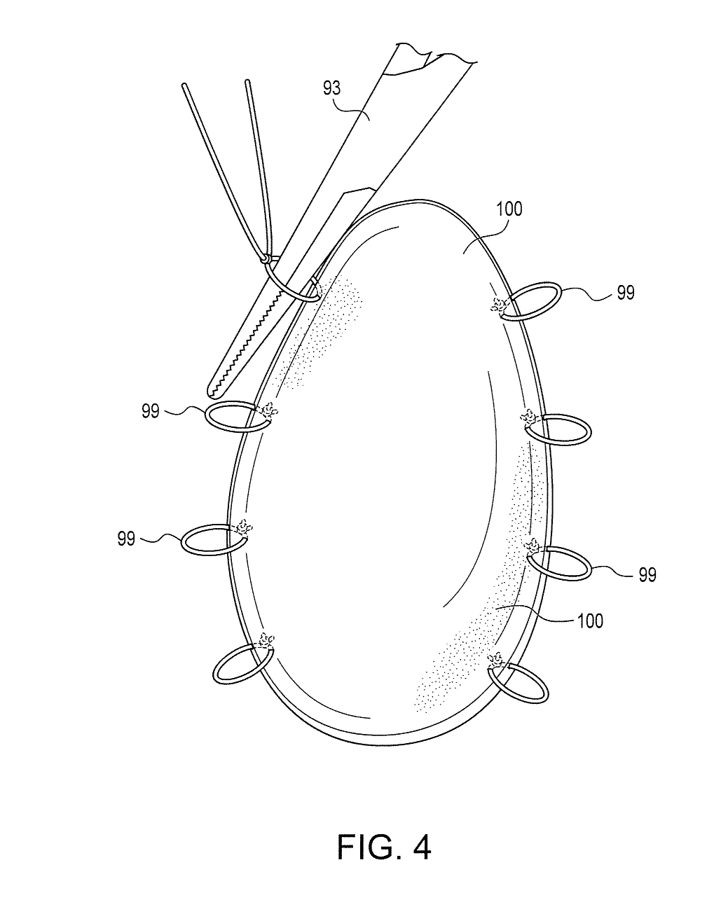

Reference is now made to FIGS. 3 and 4 which illustrate the preparation of collagen patch (graft) 100 of the present invention. Preparation of the collagen patch may be conducted simultaneously with the arthroscopic preparation of the glenoid, or at different times.

Collagen patch 100 is cut to an appropriate size necessary to cover the articular surface being repaired. The dimensions of the patch are adjusted until the dimensions of the graft closely match the dimensions of the glenoid. The preferred patch material includes a cellular bovine collagen patch that is very tear resistant. The patch optionally can be soaked or otherwise treated to infuse or coat the patch with platelet rich plasma (PRP), growth factors, medicines, cancer treatments, diagnostic markers, etc. A preferred growth factor treatment includes application of a platelet-rich plasma substance derived from plasma that has been obtained from the patient, thereby reducing the risk of autoimmune tissue rejection, such as disclosed in co-pending U.S. Patent Publication No. 2006/0085003, filed Oct. 3, 2005.

Subsequent to the dimensioning and optional treatment of the patch (graft) 100, a series of loops 99 (FIG. 4), for example suture loops, are installed along the edge of the graft 100 that will correspond to the holes 22 on the glenoid surface. The loops 99 preferably include multiple loops of a high strength suture. Exemplary high strength suture materials include, without limitation, those that are based on polyethylene. These suture materials typically contain filaments, fibers, or threads made in whole or in part from a polyethylene. A preferred example of a high strength suture is FiberWire™ suture, sold by Arthrex, Inc. of Naples, Fla., and described in U.S. Pat. No. 6,716,234, the disclosure of which is incorporated herein by reference.

Loops 99 can be formed by joining two ends of the suture, for example. The ends can be joined using knot-tying, for example. Other methods of forming loops and joining suture are contemplated by this invention. The invention also includes fabricating suture into continuous loops by “threading” at least one suture strand at least partially around the glenoid rim. The suture loops are secured preferably around the periphery of the patch 100 and, as described below, are fastened to bone using bone anchors to hold the patch in place proximate the joint surface being repaired.

As described below, loops 99 will be captured by using devices such as individual forked anchors, for example V-Tak anchors, sold by Arthrex, Inc. of Naples, Fla. and described in U.S. Patent Publ. No 2005/0080455 A1 (the disclosure of which is herein incorporated by reference), allowing attachment of the entire perimeter of the patch to the articular surface. Although FIG. 4 illustrates a total of eight loops installed in a pattern similar to that of the drill holes around the glenoid, to match hole placement for anchoring convenience, the invention contemplates any number of loops and corresponding holes formed around the glenoid. If desired, uniformity of each of the individual loops may be provided by tying over a Kelley clamp 93 (FIG. 4), for example, which is utilized as a spacer. Preferably, each loop has a diameter of about 10 to 12 mm. However, the loops may also have various diameters and, depending on the repair site, the loops may need not be uniform. Preferably, each knot corresponding to each loop is 4 throw reverse half hitches for stability. After each loop is tied, the knots may be directed to the back surface of the patch by tugging on the loops gently with a smooth grasper of forceps.

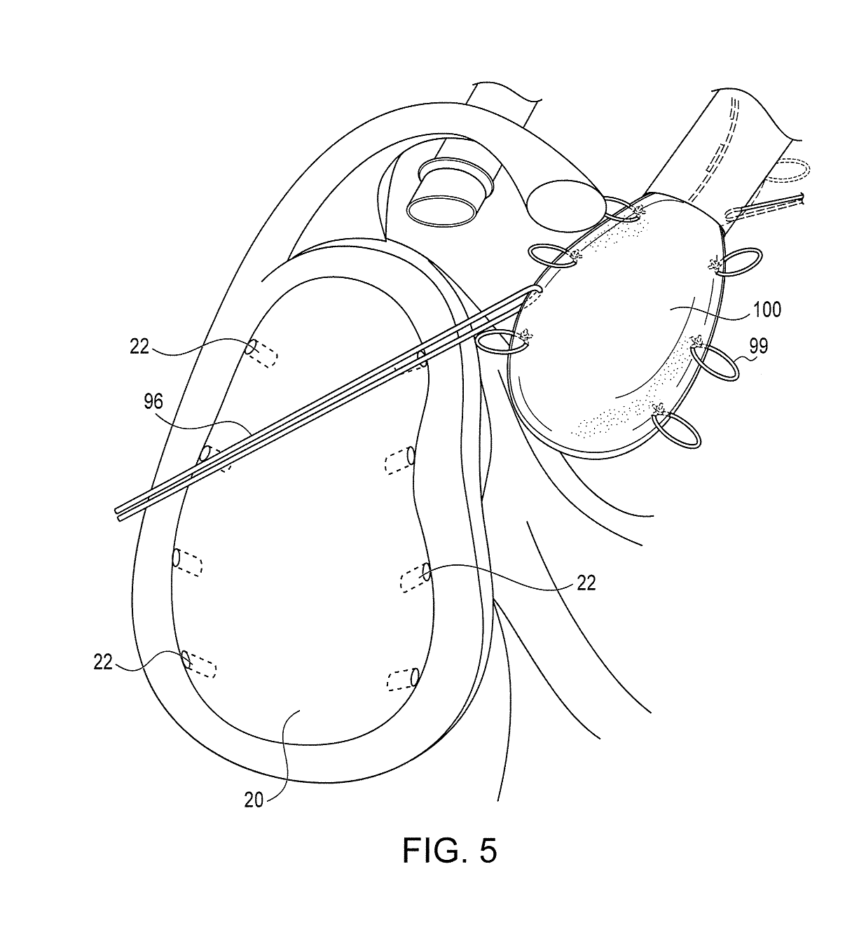

If desired, sutures 96 (FIG. 5), for example #2 FiberWire™, may be additionally employed as traction sutures on the anterior and posterior side of the patch, to aid in the introduction of the patch into the glenohumeral joint. Depending upon the side being operated upon (right versus left), each stitch is placed either above or below the midline of the graft face on its upper edge. Generally, for a left shoulder, the anterior traction stitch is placed at the 10 o'clock area and the posterior stitch is placed at the 4 o'clock area.

According to an exemplary embodiment of the present invention, patch 100 may be soaked or otherwise treated to infuse or coat the graft with platelet rich plasma (PRP), growth factors, medicines, cancer treatments and diagnostic markers, among others.

If desired, and to aid in positioning of the patch 100 in relation to the glenoid 20, the superior, anterior and posterior surfaces of the patch as they relate to the humeral head may be marked with an “s,” “a” and “p” with a sterile marking pen.

Referring now to FIG. 5, subsequent to the preparation of the glenoid surface and of the collagen patch 100, the collagen patch is introduced into the glenohumeral joint. The sutured patch 100 may be first rolled, for example, and then inserted to the end of an arthroscopic cannula. Distal, longitudinal slots are provided in the cannula to hold suture loops 99 in place. The cannula and the patch 100 (inside cannula) are inserted into the joint space, as illustrated in FIG. 5.

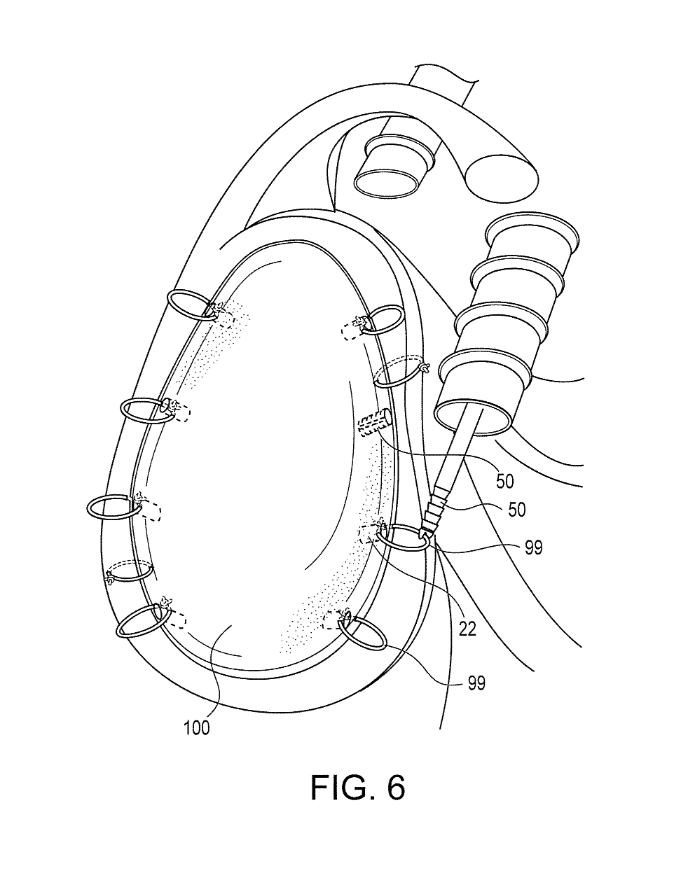

Using a grasper, the posterior #2 FiberWire™ traction sutures 96 (FIG. 5) are placed into a wire loop and the loop is pulled back through the joint from anterior to posterior, to lead the collagen patch 100 into the glenohumeral space (FIG. 6). A blunt grasper or hemostat may be utilized to facilitate patch placement into the joint from the anterior portal if the graft interferes with soft tissue during insertion.

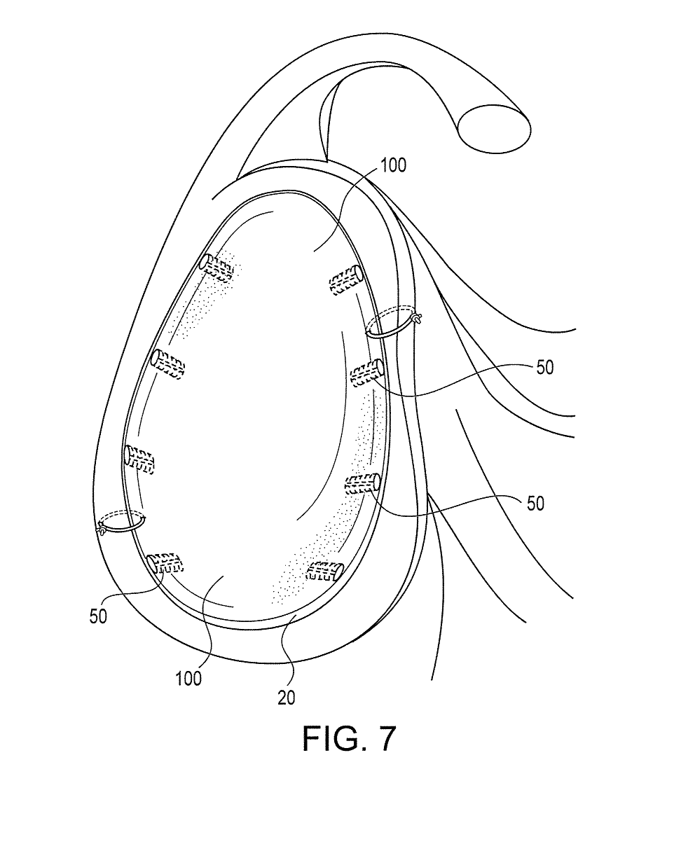

Reference is now made to FIGS. 6 and 7. After successful placement of the collagen patch 100 on the glenoid 20, the patch is anchored to the glenoid by placing the suture loops 99 in close proximity to the pre-drilled holes 22. The threaded cannula is replaced into the anterior-inferior portal for anchor placement. Suture anchors 50, for example V-Tak anchors, are installed over the loops 99 to attach the perimeter of the patch to the glenoid through the pre-drilled holes 22. Each V-Tak anchor may be introduced into the glenohumeral space through the most convenient portal/cannula and advanced down over suture loop 99, locking the suture loop into the forked tip of the V-Tak (FIG. 7). The V-Tak/suture loop combination is then positioned over the nearest corresponding drill hole 22 and advanced into the hole using a mallet, for example, until the anchor is countersunk just under the cortex and the soft tissue graft properly tensioned to the glenoid without over tensioning it. The steps may be repeated until all available and accessible suture loops 99 have been interfaced with a V-Tak and inserted into their corresponding drill holes 22 to complete the peripheral attachment of the collagen patch to the glenoid.

If further reinforcement is necessary, additional sutures may be placed around the labrum and through the upper edge of the patch using small diameter cannulated suture passing devices, such as Arthrex Micro Suture Lassos or spinal needles.

Although the present invention has been described above with reference to an exemplary glenoid repair, the invention is not limited to this exemplary embodiment. Those skilled in the art will recognize that the structures and techniques disclosed can be used for joint repair or articular surfaces generally. In addition, although the present invention has been described in connection with preferred embodiments, many modifications and variations will become apparent to those skilled in the art. Thus, it is not intended that the present invention be limited to the illustrated embodiments.

What is claimed is:

1. A graft for articular joint repair at a joint repair site, comprising:
a replacement material comprising collagen; and
sutures secured to outer edges of the replacement material.

2. The graft of claim 1, wherein the sutures are formed as loops.

3. The graft of claim 2, wherein the loops are formed at first predefined positions on the outer edges of the replacement material, the first predefined positions corresponding to second predefined positions of a plurality of holes formed on the joint repair site.

4. The graft of claim 3, wherein the loops are secured to the plurality of holes formed on the joint repair site by at least one fixation device.

5. The graft of claim 4, wherein the fixation device is an anchor with a forked tip.

6. The graft of claim 1, wherein the replacement material is cellular bovine collagen.

7. The graft of claim 1, wherein the replacement material is impregnated with growth factors, PRP, medicine, cancer treatment or diagnostic markers.

8. The graft of claim 1, wherein the replacement material has dimensions which are about equal to the dimensions of the joint repair site.

9. The graft of claim 1, wherein the joint repair site is a glenohumeral joint.

10. A graft for articular joint repair at a joint repair site, comprising:
a collagen patch; and
a material strand attached to outer edges of the collagen patch.

11. The graft of claim 10, wherein the material strand is a suture.

12. The graft of claim 11, wherein the suture is formed as loops.

13. The graft of claim 12, wherein the loops are formed at first predefined positions on the outer edges of the collagen patch, the first predefined positions corresponding to second predefined positions of a plurality of holes formed on the joint repair site.

14. The graft of claim 13, wherein the loops are secured to the plurality of holes formed on the joint repair site by at least one fixation device.

15. A method for arthroscopic joint resurfacing, comprising:
securing at least one suture to a collagen patch;
aligning the collagen patch to a joint surface in need of repair; and
fixing the suture to bone to secure the collagen patch to the joint surface.

16. The method of claim 15, wherein the step of securing the suture to the collagen patch comprises forming at least one suture loop.

17. The method of claim 16, wherein the step of fixing the suture comprises capturing the suture loop using a bone anchor.

18. The method of claim 16, wherein the step of fixing the suture comprises anchoring the suture in holes formed in the joint.

19. The method of claim 18, wherein the step of anchoring further comprises capturing the suture with a bone anchor, and controlling insertion depth of the anchor to obtain proper tensioning of the patch.

20. The method of claim 15, further comprising introducing biological factors to the patch prior to aligning the patch to the joint surface.

21. The method of claim 15, further comprising introducing biological factors to the patch prior to inserting the patch in the joint.

22. The method of claim 15, further comprising unrolling the patch across the joint surface.

23. A method of resurfacing a glenoid repair site on a glenoid, comprising:
securing sutures to a collagen patch; forming holes on the glenoid rim;
aligning the collagen patch to the glenoid repair site; and
fixing the sutures to the holes to secure the collagen patch to the glenoid.

24. The method of claim 23, wherein the step of securing sutures to the meniscal allograft comprises:
forming at least one suture loop on the collagen patch; and
capturing the at least one suture loop using a bone anchor and anchoring the at least one suture loop in the holes.

25. The method of claim 23, further comprising introducing biological factors to the glenoid repair site prior to aligning the collagen patch to the glenoid.
